(19) United States
(12) Patent Application Publication
(10) Pub. No.: US 2017/0240535 A1
(43) Pub. Date: Aug. 24, 2017

(54) HDAC1/2 INHIBITORS FOR THE TREATMENT OF NEURODEGENERATIVE AND/OR COGNITIVE DISORDERS

(71) Applicant: Regenacy Pharmaceuticals, LLC, Boston, MA (US)

(72) Inventors: John H. van Duzer, Georgetown, MA (US); Olga Golonzhka, Watertown, MA (US); Matthew B. Jarpe, Quincy, MA (US)

(21) Appl. No.: 15/422,264

(22) Filed: Feb. 1, 2017

Related U.S. Application Data
(60) Provisional application No. 62/289,675, filed on Feb. 1, 2016.

(51) Int. Cl. C07D 409/14; C07D 401/14; C07D 217/26

(52) U.S. Cl. CPC C07D 409/14; C07D 217/26; C07D 401/14

(57) ABSTRACT

Provided herein are compounds, pharmaceutical compositions comprising such compounds, and methods of using such compounds to treat or prevent diseases or disorders associated with HDAC activity, particularly diseases or disorders that involve activity of HDAC1 and/or HDAC2.

### RELATED APPLICATION

This application claims priority to U.S. Provisional Application No. 62/289,675, filed Feb. 1, 2016, which is incorporated herein by reference in its entirety.

### BACKGROUND

Alzheimer's Disease (AD) is a leading cause of dementia, and cognitive decline is one of the earlier signs of the disease. No disease-modifying treatments exist and current therapies aimed at improving cognitive function offer only marginal benefit in clinical outcomes.

Histones are highly alkaline proteins found in eukaryotic cell nuclei. Histone acetylation plays an important role in cellular processes involved in learning and memory. Histone deacetylases (HDACs) are enzymes that remove the acetyl group from an ε-N-acetyl lysine amino acid on a histone. Inhibition of histone deacetylases (HDACs) has been shown to be beneficial at improving cognitive deficits in models of neurodegeneration by enhancing expression of synaptic plasticity genes through increase of histone acetylation at gene promoters. It has been proposed that dysregulation of histone acetylation contributes to the cognitive impairments associated with AD and HDAC inhibitors provide an exciting avenue for developing novel therapeutics option to address cognitive decline in AD patients.

Further, it has been shown that HDAC2 expression and activity are elevated in neurodegenerative diseases (Guan et al., “HDAC2 negatively regulates memory formation and synaptic plasticity,” Nature 2009, 459 (7234), 55-60; Morris et al., “Loss of histone deacetylase 2 improves working memory and accelerates extinction learning,” J. Neurosci. 2013 33(15) 6401-64011). Increasing the expression of HDAC2 impairs cognitive function in mice. Inhibition of HDAC2 by gene disruption restores cognitive function in mouse models of Alzheimer's disease (Guan et al., 2009; Morris et al., 2013; Graff et al., “Epigenetic Priming of Memory Updating during Reconsolidation to Attenuate Remote Fear Memories,” Cell 2014, 15(0), 261-276)).

Inhibition of HDAC1 and HDAC2 has also been shown to derepress fetal globin. Fetal hemoglobin (HbF) derepression, or induction, is an established therapeutic strategy in sickle cell disease, and could also be effective in treating beta-thalassemia. Hydroxyurea is currently the only drug with proven efficacy in sickle cell disease (SCD). This therapy is cytotoxic, poorly tolerated, and only reduces the frequency and severity of sickle cell crises in a subset of patients. There are no approved drugs for the treatment of beta-thalassemia. Fetal (γ) globin expression is silenced in adults partly through the action of a complex containing BCL11A and HDACs 1 and 2. Genetic ablation and chemical inhibition of HDAC1 or HDAC2 results in the derepression of γ globin in adult bone marrow derived erythroid cells (Bradner, Proc. Natl. Acad. Sci. 2010). While a variety of non-specific HDAC inhibitors have been used successfully to induce HbF, further clinical development has been limited by their variable efficacy and concerns over off target side-effects observed in small clinical trials. Therefore, development of selective and potent HDAC1 and HDAC2 inhibitors leading to HbF reactivation represents a refined and more targeted therapeutic approach for the treatment of SCD and beta-thalassemia.

It has also been shown that deregulated HDAC1 expression is particularly common in advanced cancers of the gastrointestinal system, such as, for example, pancreatic, colorectal, and liver (hepatocellular) carcinomas, as well as in prostate and breast cancer. HDAC2 and HDAC3 expression are also associated with advanced stage disease and poor prognosis in gastric, prostate and colorectal cancers. HDAC2 is also over expressed in cervical cancer. Clinical trials for the treatment of patients with advanced solid tumors, lymphomas, and leukemias utilizing class I selective HDAC inhibitors such as MS275, depsipeptide, and MGCD0103 have been published (O. Witt et al., Cancer Letters, 2009, 277, 8-21 and H-J. Kim and S.-C. Bae, Am. J. Transl. Res. 2011; 3(2): 166-179).

There remains a need for preparing structurally diverse HDAC inhibitors, particularly ones that are potent and/or selective inhibitors of particular classes of HDACs and individual HDACs (e.g., HDAC1 and/or HDAC2).

### SUMMARY

Provided herein are compounds, pharmaceutical compositions comprising such compounds, and methods of using such compounds to treat or prevent diseases or disorders associated with HDAC activity, particularly diseases or disorders that involve any type of HDAC1 and/or HDAC2 activity. Such diseases include, but are not limited to, cancer, disorders of the central nervous system (e.g., neurodegenerative disorders or diseases), sickle-cell anemia, and beta-thalassemia.

Thus, in one aspect, provided herein is a compound of Formula I

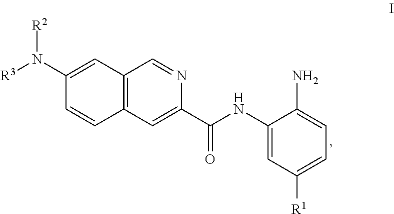

or a pharmaceutically acceptable salt thereof.

In another aspect, provided herein is a compound of Formula IA

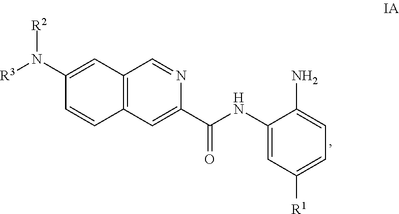

or a pharmaceutically acceptable salt thereof.

In an embodiment, provided herein is a compound of Formula II:

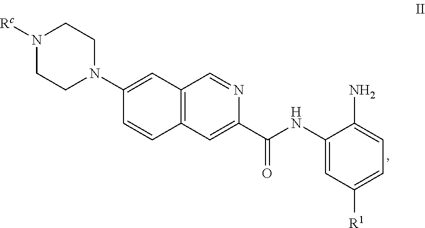

or a pharmaceutically acceptable salt thereof.

In an embodiment, provided herein are the compounds of Table 1, or pharmaceutically acceptable salts thereof.

In another aspect, provided herein is a pharmaceutical composition comprising a compound of Formula I, a compound of Formula IA, a compound of Formula II, a compound present in Table 1, or a pharmaceutically acceptable salt thereof, and at least one pharmaceutically acceptable carrier.

In another aspect, provided herein is a method for selectively inhibiting histone deacetylase 1/2 (HDAC1/2) in a cell comprising contacting said cell with a compound of Formula I, a compound of Formula IA, a compound of Formula II, a compound present in Table 1, or a pharmaceutically acceptable salt thereof, and at least one pharmaceutically acceptable carrier.

In another aspect, provided herein is a method for treating a disease associated with HDAC1/2 activity in a subject in need thereof comprising administering to the subject a therapeutically effective amount of a compound of Formula I, a compound of Formula IA, a compound of Formula II, a compound present in Table 1, or a pharmaceutically acceptable salt thereof.

In another aspect, provided herein is a method for treating a neurodegenerative disorder in a subject in need thereof comprising administering to the subject a therapeutically effective amount of a compound of Formula I, a compound of Formula IA, a compound of Formula II, a compound present in Table 1, or a pharmaceutically acceptable salt thereof.

In an embodiment, the neurodegenerative disorder is characterized by cognitive dysfunction.

In a further embodiment, the neurodegenerative disorder is selected from the group consisting of Alzheimer's disease, Huntington's disease, frontotemporal dementia, progressive supranuclear palsy, corticobasal dementia, Parkinson's with Lewy-Body dementia, post-traumatic neurodegeneration, and chronic traumatic encephalopathy.

In another embodiment, the neurodegenerative disorder is not characterized by cognitive dysfunction.

In a further embodiment, the neurodegenerative disorder is selected from the group consisting of Parkinson's disease, amyotrophic lateral sclerosis (ALS), and multiple sclerosis (MS).

In an aspect, provided herein is a method for treating or preventing a condition or disorder characterized by cognitive dysfunction in a subject in need thereof comprising administering to the subject a therapeutically effective amount of a compound of Formula I, a compound of Formula IA, a compound of Formula II, a compound present in Table 1, or a pharmaceutically acceptable salt thereof.

In an embodiment, the condition or disorder characterized by cognitive dysfunction is selected from the group consisting of schizophrenia, cognitive impairment associated with ischemic events, depression, and posttraumatic stress disorder (PTSD).

In another aspect, provided herein is a method for treating bipolar disorder in a subject in need thereof comprising administering to the subject a therapeutically effective amount of a compound of Formula I, a compound of Formula IA, a compound of Formula II, a compound present in Table 1, or a pharmaceutically acceptable salt thereof.

In an aspect, provided herein is a method for treating or preventing Alzheimer's Disease in a subject in need thereof comprising administering to the subject a therapeutically effective amount of a compound of Formula I, a compound of Formula IA, a compound of Formula II, a compound present in Table 1, or a pharmaceutically acceptable salt thereof.

In another aspect, provided herein is a method for treating Alzheimer's Disease in a subject in need thereof comprising administering to the subject a therapeutically effective amount of a compound of Formula I, a compound of Formula IA, a compound of Formula II, a compound present in Table 1, or a pharmaceutically acceptable salt thereof.

In another aspect, provided herein is a method for enhancing cognition in a subject with Alzheimer's Disease comprising administering to the subject a compound of Formula I, a compound of Formula IA, a compound of Formula II, a compound present in Table 1, or a pharmaceutically acceptable salt thereof.

In another aspect, provided herein is a method for enhancing memory in a subject with Alzheimer's Disease comprising administering to the subject a compound of Formula I, a compound of Formula IA, a compound of Formula II, a compound present in Table 1, or a pharmaceutically acceptable salt thereof.

In yet another aspect, provided herein is a method for treating or preventing Alzheimer's disease in a subject comprising administering to the subject a therapeutically effective amount of a histone deacetylase 1/2 (HDAC1/2) selective inhibitor.

In another aspect, provided herein is a method for enhancing cognition in a subject with Alzheimer's Disease comprising administering to the subject a histone deacetylase 1/2 (HDAC1/2) selective inhibitor.

In another aspect, provided herein is a method for enhancing memory in a subject with Alzheimer's Disease comprising administering to the subject a histone deacetylase 1/2 (HDAC1/2) selective inhibitor.

### BRIEF DESCRIPTION OF THE FIGURES

FIG. 1A and FIG. 1B show graphs representing the performance of wild type and APPSwDI/NOS2−/− mice (treated with vehicle or 10 mg/kg compound 1 for 4 weeks) in a radial arm water maze. FIG. 1A shows the escape latency in seconds, and FIG. 1B shows the number of errors the mice made in searching for the escape platform. Data are presented as mean±SEM. Asterisks indicate a significant different in escape latency in seconds and the number of errors between the wild type and transgenic mice, whereas the transgenic mice treated with compound 1 are not statistically different from wild type mice. (See Example 3).

FIG. 2 shows graphs representing the performance of young (3 months old) and aged (18 months old) mice in a Morris Water Maze cued and place tests. The left graph shows the escape latency in seconds of mice when cued to the location of the platform. There is no significant difference between the performance of young and aged mice in this test. The right graphs shows the escape latency in seconds in the place test in which the escape platform is hidden. Young mice show improved escape latency with repeated trials, while aged mice do not. Young and aged mice treated with compound 1 (10 mg/kg/day) for 4 weeks perform better in the place test than the corresponding untreated mice. (See Example 4).

### DETAILED DESCRIPTION

A biological target of current interest is histone deacetylase (HDAC) (see, for example, a discussion of the use of inhibitors of histone deacetylases for the treatment of cancer: Marks et al. Nature Reviews Cancer 2001, 7, 194; Johnstone et al. Nature Reviews Drug Discovery 2002, 287). Post-translational modification of proteins through acetylation and deacetylation of lysine residues plays a critical role in regulating their cellular functions. HDACs are zinc hydrolases that modulate gene expression through deacetylation of the N-acetyl-lysine residues of histone proteins and other transcriptional regulators (Hassig et al. Curr. Opin. Chem. Biol. 1997, 1, 300-308). HDACs participate in cellular pathways that control cell shape and differentiation, and an HDAC inhibitor has been shown to be effective in treating an otherwise recalcitrant cancer (Warrell et al. J. Natl. Cancer Inst. 1998, 90, 1621-1625).

Eleven human HDACs, which use Zn as a cofactor, have been identified (Taunton et al. Science 1996, 272, 408-411; Yang et al. J. Biol. Chem. 1997, 272, 28001-28007. Grozinger et al. Proc. Natl. Acad. Sci. U.S.A. 1999, 96, 4868-4873; Kao et al. Genes Dev. 2000, 14, 55-66. Hu et al. J. Biol. Chem. 2000, 275, 15254-15264; Zhou et al. Proc. Natl. Acad. Sci U.S.A. 2001, 98, 10572-10577; Venter et al. Science 2001, 291, 1304-1351) and these members fall into three classes (class I, II, and IV) based on sequence homology to their yeast orthologues (O. Witt et al. Cancer Letters, 2009, 277, 8-21). Class I HDACs include HDAC1, HDAC2, HDAC3, and HDAC8, and are referred to as “classical” HDACs, which implies a catalytic pocket with a Zn²⁺ ion at its base.

Accordingly, provided herein are compounds, pharmaceutical compositions comprising such compounds, and methods of using such compounds to treat or prevent diseases or disorders associated with HDAC activity, particularly diseases or disorders that involve any type of HDAC1 and/or HDAC2 activity. Such diseases include, but are not limited to, cancer, disorders of the central nervous system (e.g., neurodegenerative disorders or diseases), sickle-cell anemia, and beta-thalassemia.

Further, provided herein is a class of HDAC1/2-selective compounds with unique blood brain barrier penetration properties. Thus, the compounds provided herein are particularly suitable for treating central nervous system disorders and/or disease characterized by cognitive dysfunction. These compounds can provide sustained high brain to plasma exposure ratios, which allows for maximum activity in the target tissue and minimizes the toxicity in the periphery known to be associated with HDAC1/2 inhibition.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

The number of carbon atoms in an alkyl substituent can be indicated by the prefix “Cₓ-C_{y},” where x is the minimum and y is the maximum number of carbon atoms in the substituent. Likewise, a Cₓ chain means an alkyl chain containing x carbon atoms.

The term “about” generally indicates a possible variation of no more than 10%, 5%, or 1% of a value. For example, “about 25 mg/kg” will generally indicate, in its broadest sense, a value of 22.5-27.5 mg/kg, i.e., 25±2.5 mg/kg.

The term “alkyl” refers to saturated, straight- or branched-chain hydrocarbon moieties containing, in certain embodiments, between one and six, or one and eight carbon atoms, respectively. The number of carbon atoms in an alkyl substituent can be indicated by the prefix “Cₓ-C_{y},” where x is the minimum and y is the maximum number of carbon atoms in the substituent. Likewise, a Cₓ chain description indicates a group containing x carbon atoms (i.e., not including the number of heteroatoms). Examples of C₁-C₆-alkyl moieties include, but are not limited to, methyl, ethyl, propyl, isopropyl, n-butyl, tert-butyl, neopentyl, n-hexyl moieties; and examples of C₁-C₈-alkyl moieties include, but are not limited to, methyl, ethyl, propyl, isopropyl, n-butyl, tert-butyl, neopentyl, n-hexyl, heptyl, and octyl moieties.

The term “aryl” refers to a mono- or poly-cyclic carbocyclic ring system having one or more aromatic rings, fused or non-fused, including, but not limited to, phenyl (i.e., C₆-aryl), naphthyl, tetrahydronaphthyl, indanyl, idenyl, and the like. In some embodiments, aryl groups have 6 carbon atoms (e.g., C₆-aryl or phenyl). In some embodiments, aryl groups have from six to ten carbon atoms (e.g., C₆-C₁₀-aryl). In some embodiments, aryl groups have from six to sixteen carbon atoms.

The term “heteroaryl” refers to a mono- or poly-cyclic (e.g., bi-, or tri-cyclic or more) fused or non-fused moiety or ring system having at least one aromatic ring, where one or more of the ring-forming atoms is a heteroatom such as oxygen, sulfur, or nitrogen. In some embodiments, the heteroaryl group has one to eight carbon atoms, one to six carbon atoms, two to 6 carbon atoms (e.g., C₁-C₈-heteroaryl, C₁-C₆-heteroaryl, or C₂-C₆-heteroaryl). In further embodiment the heteroaryl group has one to fifteen carbon atoms. In some embodiments, the heteroaryl group contains five to sixteen ring atoms of which one ring atom is selected from oxygen, sulfur, and nitrogen; zero, one, two, or three ring atoms are additional heteroatoms independently selected from oxygen, sulfur, and nitrogen; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiazolyl, thiadiazolyl, oxadiazolyl, thiophenyl, furanyl, indolyl, quinolinyl, isoquinolinyl, benzimidazolyl, benzooxazolyl, quinoxalinyl, acridinyl, and the like.

The term “heterocycloalkyl” refers to a non-aromatic 3-, 4-, 5-, 6- or 7-membered ring or a bi- or tri-cyclic group fused of non-fused system, where (i) each ring contains between one and three heteroatoms independently selected from oxygen, sulfur, and nitrogen and the remaining atoms are carbon (e.g., C₂-C₆-heterocyclyl, C₃-C₆-heterocyclyl, or C₃-C₅-heterocyclyl), (ii) each 5-membered ring has 0 to 1 double bonds and each 6-membered ring has 0 to 2 double bonds, (iii) the nitrogen and sulfur heteroatoms can optionally be oxidized, (iv) the nitrogen heteroatom can optionally be quaternized, and (iv) any of the above rings can be fused to a benzene ring. The term “heterocycloalkyl” includes, but is not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

The term “inhibitor” is synonymous with the term antagonist.

The term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Additionally, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

### Compounds of the Invention

Provided herein is a compound of Formula I

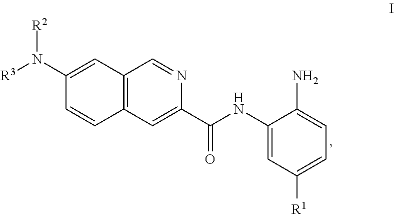

or a pharmaceutically acceptable salt thereof,

wherein

R¹ is aryl or heteroaryl;

R² is H, C₁-C₆-alkyl, or C₁-C₆-alkyl-N(R^{a})₂;

R³ is H, C₁-C₆-alkyl, C₁-C₆-alkyl-N(R^{a})₂, or C(O)R^{b};

or R² and R³, together with the N atom to which they are attached, optionally form a 5 or 6 membered heterocycloalkyl ring, wherein the heterocycloalkyl ring optionally contains a —N(R^{c})— moiety and wherein the heterocycloalkyl ring optionally contains a —C(O)— moiety;

each R^{a} is independently H or C₁-C₆-alkyl;

R^{b} is C₁-C₆-alkyl, C₁-C₆-alkyl-N(R^{d})₂, or a 5 or 6 membered heterocycloalkyl, wherein the heterocycloalkyl is optionally substituted by C₁-C₆-alkyl;

R^{c} is H or C₁-C₆-alkyl; and

each R^{d} is independently H or C₁-C₆-alkyl.

In an embodiment of the compound of Formula I, R¹ is phenyl or a 5 or 6 membered heteroaryl containing one or more heteroatoms independently selected from O, N, and S.

In an embodiment of the compound of Formula I, R¹ is phenyl, thiophenyl, or pyridinyl.

In a further embodiment of the compound of Formula I, R² is H and R³ is C₁-C₆-alkyl-N(R^{a})₂.

In yet another embodiment of the compound of Formula I,

R² is H or C₁-C₆-alkyl;

R³ is C₁-C₆-alkyl-N(R^{a})₂ or C(O)R^{b};

or R² and R³, together with the N atom to which they are attached, optionally form a 5 or 6 membered heterocycloalkyl ring, wherein the heterocycloalkyl ring optionally contains a —C(O)— moiety and wherein the heterocycloalkyl ring optionally contains a —N(R^{c})— moiety.

In a further embodiment, R^{b} is pyrrolidine optionally substituted by C₁-C₆-alkyl.

In another embodiment of the compound of Formula I, R² and R³, together with the N atom to which they are attached, form a 5 or 6 membered heterocycloalkyl ring, wherein the heterocycloalkyl ring optionally contains a —N(R^{c})— moiety and wherein the heterocycloalkyl ring optionally contains a —C(O)— moiety.

In another embodiment of the compound of Formula I, R² and R³, together with the N atom to which they are attached, form a 5 or 6 membered heterocycloalkyl ring, wherein the heterocycloalkyl ring contains a —N(R^{c})— moiety and wherein the heterocycloalkyl ring optionally contains a —C(O)— moiety.

In an embodiment, the compound of Formula I is a compound of Formula II:

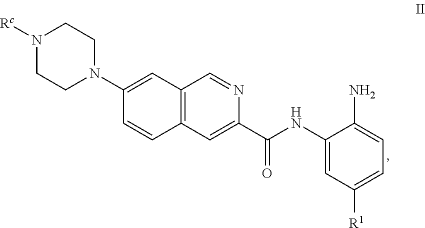

or a pharmaceutically acceptable salt thereof,

wherein

R¹ is aryl or heteroaryl; and

R^{c} is H or C₁-C₆-alkyl.

In an embodiment of the compound of Formula II, R¹ is phenyl or a 5 or 6 membered heteroaryl containing one or more heteroatoms independently selected from O, N, and S.

In an embodiment of the compound of Formula II, R¹ is phenyl, thiophenyl, or pyridinyl.

In another embodiment of the compound of Formula II, R¹ is phenyl or thiophenyl.

In an embodiment of the compound of Formula II, R^{c} is H.

Provided herein is a compound of Formula IA

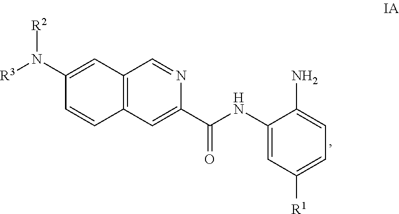

or a pharmaceutically acceptable salt thereof,

wherein

R¹ is aryl that is substituted one or more times with halo;

R² is H, C₁-C₆-alkyl, or C₁-C₆-alkyl-N(R^{a})₂;

R³ is H, C₁-C₆-alkyl, C₁-C₆-alkyl-N(R^{a})₂, or C(O)R^{b};

or R² and R³, together with the N atom to which they are attached, optionally form a 5 or 6 membered heterocycloalkyl ring, wherein the heterocycloalkyl ring optionally contains a —N(R^{c})— moiety and wherein the heterocycloalkyl ring optionally contains a —C(O)— moiety;

each R^{a} is independently H or C₁-C₆-alkyl;

R^{b} is C₁-C₆-alkyl, C₁-C₆-alkyl-N(R^{d})₂, or a 5 or 6 membered heterocycloalkyl, wherein the heterocycloalkyl is optionally substituted by C₁-C₆-alkyl;

R^{c} is H or C₁-C₆-alkyl; and

each R^{d} is independently H or C₁-C₆-alkyl.

In an embodiment of the compound of Formula IA, R¹ is phenyl that is substituted one or more times with halo.

In an embodiment of the compound of Formula I, R¹ is phenyl that is substituted by fluoro.

In a further embodiment of the compound of Formula IA, R² is H and R³ is C₁-C₆-alkyl-N(R^{a})₂.

In yet another embodiment of the compound of Formula IA,

R² is H or C₁-C₆-alkyl;

R³ is C₁-C₆-alkyl-N(R^{a})₂ or C(O)R^{b};

or R² and R³, together with the N atom to which they are attached, optionally form a 5 or 6 membered heterocycloalkyl ring, wherein the heterocycloalkyl ring optionally contains a —C(O)— moiety and wherein the heterocycloalkyl ring optionally contains a —N(R^{c})— moiety.

In a further embodiment of Formula IA, R^{b} is pyrrolidine optionally substituted by C₁-C₆-alkyl.

In another embodiment of the compound of Formula IA, R² and R³, together with the N atom to which they are attached, form a 5 or 6 membered heterocycloalkyl ring, wherein the heterocycloalkyl ring optionally contains a —N(R^{c})— moiety and wherein the heterocycloalkyl ring optionally contains a —C(O)— moiety.

In another embodiment of the compound of Formula IA, R² and R³, together with the N atom to which they are attached, form a 5 or 6 membered heterocycloalkyl ring, wherein the heterocycloalkyl ring contains a —N(R^{c})— moiety and wherein the heterocycloalkyl ring optionally contains a —C(O)— moiety.

In another aspect, provided herein is a compound selected from any of the compounds presented in Table 1:

TABLE 1

| | |
|---|---|
| 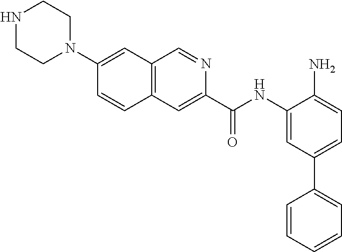 [SMILES: Nc1ccc(-c2ccccc2)cc1NC(=O)c1cc2ccc(N3CCNCC3)cc2cn1] | Compound 1 |
| 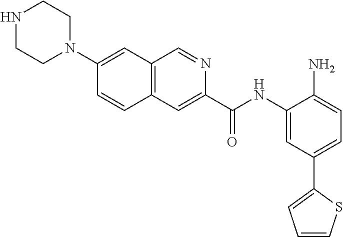 [SMILES: Nc1ccc(-c2cccs2)cc1NC(=O)c1cc2ccc(N3CCNCC3)cc2cn1] | Compound 2 |
| 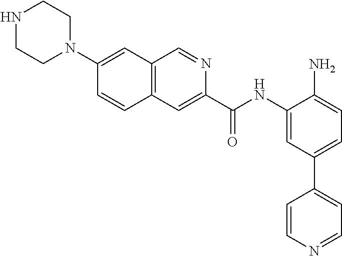 [SMILES: Nc1ccc(-c2ccncc2)cc1NC(=O)c1cc2ccc(N3CCNCC3)cc2cn1] | Compound 3 |
| 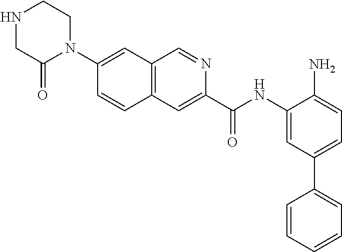 [SMILES: Nc1ccc(-c2ccccc2)cc1NC(=O)c1cc2ccc(N3CCNCC3=O)cc2cn1] | Compound 4 |
| 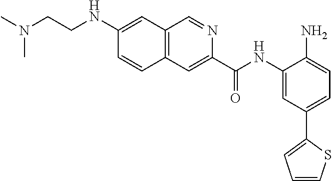 [SMILES: CN(C)CCNc1ccc2cc(C(=O)Nc3cc(-c4cccs4)ccc3N)ncc2c1] | Compound 5 |
| 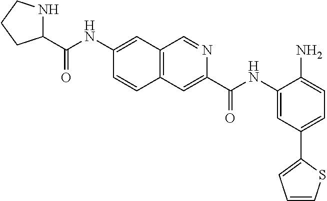 [SMILES: Nc1ccc(-c2cccs2)cc1NC(=O)c1cc2ccc(NC(=O)C3CCCN3)cc2cn1] | Compound 6 |
| 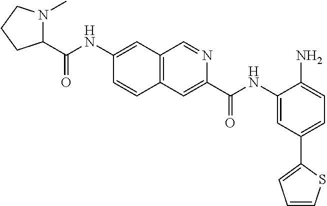 [SMILES: CN1CCCC1C(=O)Nc1ccc2cc(C(=O)Nc3cc(-c4cccs4)ccc3N)ncc2c1] | Compound 7 |
| 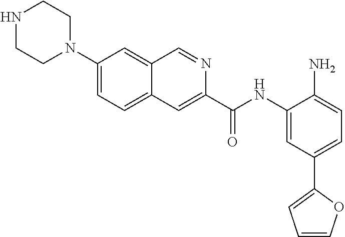 [SMILES: Nc1ccc(-c2ccco2)cc1NC(=O)c1cc2ccc(N3CCNCC3)cc2cn1] | Compound 8 |
| 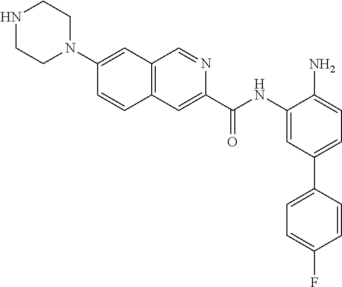 [SMILES: Nc1ccc(-c2ccc(F)cc2)cc1NC(=O)c1cc2ccc(N3CCNCC3)cc2cn1] | Compound 9 |

and pharmaceutically acceptable salts thereof.

Compounds of Formula I, compounds of Formula IA, compounds of Formula II, compounds of Table 1, and pharmaceutically acceptable salts thereof, are referred to herein as “compounds of the invention.”

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

In one embodiment, the compounds of the invention may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and related compounds having different substituents can be synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein can be synthesized using any suitable procedures starting from compounds that are available from commercial sources, or can be prepared using procedures described herein (See Example 1, which described the synthesis of Compounds 1 and 2).

### Pharmaceutical Compositions

Provided herein is a pharmaceutical composition comprising a compound of the invention (including pharmaceutically acceptable salts thereof), and at least one pharmaceutically acceptable carrier.

In an aspect, provided herein is a pharmaceutical composition comprising a compound of the Formula I, or a pharmaceutically acceptable salt thereof, and at least one pharmaceutically acceptable carrier.

In another aspect, provided herein is a pharmaceutical composition comprising a compound of the Formula IA, or a pharmaceutically acceptable salt thereof, and at least one pharmaceutically acceptable carrier.

In another aspect, provided herein is a pharmaceutical composition comprising a compound of Formula II, or a pharmaceutically acceptable salt thereof, and at least one pharmaceutically acceptable carrier.

In another aspect, provided herein is a pharmaceutical composition comprising a compound of Table 1, or a pharmaceutically acceptable salt thereof, and at least one pharmaceutically acceptable carrier.

As used herein, the term “composition” or “pharmaceutical composition” refers to a mixture of at least one compound useful within the invention with a pharmaceutically acceptable carrier. The pharmaceutical composition facilitates administration of the compound to a subject or patient. Multiple techniques of administering a compound exist in the art including, but not limited to, intravenous, oral, aerosol, parenteral, ophthalmic, pulmonary, and topical administration.

These pharmaceutical compositions comprise a therapeutically effective amount of a compound of the present invention formulated together with one or more pharmaceutically acceptable carriers. The term “pharmaceutically acceptable carrier” means a non-toxic, inert solid, semi-solid or liquid filler, diluent, encapsulating material or formulation auxiliary of any type. The pharmaceutical compositions can be administered to humans and other animals orally, rectally, parenterally, intracisternally, intravaginally, intraperitoneally, topically (as by powders, ointments, or drops), buccally, or as an oral or nasal spray.

Compounds of the invention can be administered as pharmaceutical compositions by any conventional route, in particular enterally, for example, orally, e.g., in the form of tablets or capsules, or parenterally, e.g., in the form of injectable solutions or suspensions, topically, e.g., in the form of lotions, gels, ointments or creams, or in a nasal or suppository form.

Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose. In the case of capsules, tablets and pills, the dosage forms can also comprise buffering agents.

### Methods for Treating

Provided herein are methods for treating or preventing disorders in a subject, such as a human or other animal, by administering to the subject a therapeutically effective amount of a compound of the invention, in such amounts and for such time as is necessary to achieve the desired result. The term “therapeutically effective amount” of a compound of the invention means a sufficient amount of the compound so as to decrease the symptoms of a disorder in a subject. As is well understood in the medical arts a therapeutically effective amount of a compound of this invention will be at a reasonable benefit/risk ratio applicable to any medical treatment. The exact amount required will vary from subject to subject, depending on the species, age, and general condition of the subject, the severity of the disease, the particular active agent, its mode of administration, and the like.

The term “treating” or “treatment” as used herein comprises relieving, reducing or alleviating at least one symptom in a subject or effecting a delay of progression of a disease. For example, treatment can be the diminishment of one or several symptoms of a disorder or complete eradication of a disorder, such as Alzheimer's Disease. Within the meaning of the present disclosure, the term “treat” also denotes a reduction in the risk of worsening a disease.

The term “protect” is used herein to mean prevent, delay, or treat, or all, as appropriate, development, continuance or aggravation of a disease in a subject, e.g., a mammal or human.

The term “prevent,” “preventing” or “prevention” as used herein comprises the prevention of at least one symptom associated with or caused by the state, disease or disorder being prevented.

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors.

In certain embodiments, a therapeutic amount or dose of the compounds of the present invention can range from about 0.1 mg/kg to about 500 mg/kg (about 0.18 mg/m² to about 900 mg/m²), alternatively from about 1 to about 50 mg/kg (about 1.8 to about 90 mg/m²). In general, treatment regimens according to the present invention comprise administration to a subject in need of such treatment from about 10 mg to about 1000 mg of the compound(s) of this invention per day in single or multiple doses. Therapeutic amounts or doses will also vary depending on route of administration, as well as the possibility of co-usage with other agents.

Upon improvement of a subject's condition, a maintenance dose of a compound, composition or combination of this invention can be administered, if necessary. Subsequently, the dosage or frequency of administration, or both, can be reduced, as a function of the symptoms, to a level at which the improved condition is retained when the symptoms have been alleviated to the desired level, treatment should cease. The subject can, however, require intermittent treatment on a long-term basis upon any recurrence of disease symptoms.

It will be understood, however, that the total daily usage of the compounds and compositions of the present invention will be decided by the attending physician within the scope of sound medical judgment. The specific inhibitory dose for any particular subject (or patient) will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

As described, the compounds provided herein have HDAC1 and/or HDAC2 inhibitory activity (see, e.g., Example 2, Table 2).

Thus, in an aspect, provided herein is a method for selectively inhibiting histone deacetylase 1/2 (HDAC1/2) in a cell comprising contacting said cell with a compound of the invention (i.e., a compound of Formula I, a compound of Formula IA, a compound of Formula II, a compound of Table 1, or a pharmaceutically acceptable salt thereof).

In another aspect, provided herein is a method for treating a disease associated with HDAC1/2 activity in a subject in need thereof comprising administering to the subject a therapeutically effective amount of a compound of the invention.

In an embodiment, the disease or disorder associated with HDAC1/2 activity is selected from Alzheimer's disease, Huntington's disease, frontotemporal dementia, progressive supranuclear palsy, corticobasal dementia, Parkinson's with Lewy-Body dementia, post-traumatic neurodegeneration, chronic traumatic encephalopathy, Parkinson's disease, amyotrophic lateral sclerosis (ALS), multiple sclerosis (MS), schizophrenia, cognitive impairment associated with ischemic events, depression, posttraumatic stress disorder (PTSD), and bipolar disorder.

In another embodiment, the disease or disorder associated with HDAC1/2 activity is a hemoglobinopathy. In a preferred embodiment, the disorder is sickle-cell disease or beta-thalessemia.

In another embodiment, disease or disorder associated with HDAC1/2 activity are myelodysplastic syndromes.

In another embodiment, disease or disorder associated with HDAC1/2 activity is cancer. In particular embodiments, the cancer is lung cancer, colon and rectal cancer, breast cancer, prostate cancer, liver cancer, pancreatic cancer, brain cancer, kidney cancer, ovarian cancer, stomach cancer, skin cancer, bone cancer, gastric cancer, breast cancer, glioma, glioblastoma, neuroblastom, hepatocellular carcinoma, papillary renal carcinoma, head and neck squamous cell carcinoma, leukemia, lymphomas, myelomas, retinoblastoma, cervical cancer, melanoma and/or skin cancer, bladder cancer, uterine cancer, testicular cancer, esophageal cancer, and solid tumors. In some embodiments, the cancer is lung cancer, colon cancer, breast cancer, neuroblastoma, leukemia, or lymphomas. In other embodiments, the cancer is lung cancer, colon cancer, breast cancer, neuroblastoma, leukemia, or lymphoma. In a further embodiment, the cancer is non-small cell lung cancer (NSCLC) or small cell lung cancer. In another embodiment, the cancer is neuroblastoma.

In further embodiments, the cancer is a hematologic cancer, such as leukemia or lymphoma. In a certain embodiment, lymphoma is Hodgkins lymphoma or Non Hodgkin's lymphoma. In certain embodiments, leukemia is myeloid, lymphocytic, myelocytic, lymphoblastic, or megakaryotic leukemia. In a particular embodiment, the leukemia is acute myelogenous leukemia and megakaryocytic leukemia.

In an aspect, provided herein is a method for treating Alzheimer's disease, Huntington's disease, frontotemporal dementia, progressive supranuclear palsy, corticobasal dementia, Parkinson's with Lewy-Body dementia, post-traumatic neurodegeneration, chronic traumatic encephalopathy, Parkinson's disease, amyotrophic lateral sclerosis (ALS), multiple sclerosis (MS), schizophrenia, cognitive impairment associated with ischemic events, depression, posttraumatic stress disorder (PTSD), and bipolar disorder.

In another aspect, provided herein is a method for treating sickle cell disease, beta thalassemia, myelodysplastic syndrome, acute myelogenous leukemia, neuroblastoma, or megakaryocytic leukemia in a subject comprising administering to the subject in need thereof a therapeutically effective amount of a compound of Formula I, a compound of Formula IA, a compound of Formula II, a compound presented in Table 1, or a pharmaceutically acceptable salt of any of the foregoing compounds.

In an aspect, provided herein is a method for treating sickle cell disease, beta thalassemia, myelodysplastic syndrome, acute myelogenous leukemia, neuroblastoma, or megakaryocytic leukemia in a subject comprising administering to the subject a therapeutically effective amount of compound of Formula I, or a pharmaceutically acceptable salt thereof.

In an aspect, provided herein is a method for treating sickle cell disease, beta thalassemia, myelodysplastic syndrome, acute myelogenous leukemia, neuroblastoma, or megakaryocytic leukemia in a subject comprising administering to the subject a therapeutically effective amount of compound of Formula IA, or a pharmaceutically acceptable salt thereof.

In an aspect, provided herein is a method for treating sickle cell disease, beta thalassemia, myelodysplastic syndrome, acute myelogenous leukemia, neuroblastoma, or megakaryocytic leukemia in a subject comprising administering to the subject a therapeutically effective amount of compound of Formula II, or a pharmaceutically acceptable salt thereof.

Further, the compounds of the invention act as HDAC1/2-selective compounds with unique blood brain barrier penetration properties (see, e.g., Example 3). Thus, the compounds provided herein are particularly suitable for treating central nervous system disorders and/or cognitive disorders. These compounds can provide sustained high brain to plasma exposure ratios, which allows for maximum activity in the target tissue and minimizes the toxicity in the periphery known to be associated with HDAC1/2 inhibition (see Example 3, Table 4).

In an aspect, provided herein is a method for treating a neurodegenerative disorder in a subject in need thereof comprising administering to the subject a therapeutically effective amount of a compound of the invention.

In an embodiment, the neurodegenerative disorder is characterized by cognitive dysfunction. Non-limiting examples of neurodegenerative disorders characterized by cognitive dysfunction include Alzheimer's disease, Huntington's disease, frontotemporal dementia, progressive supranuclear palsy, corticobasal dementia, Parkinson's with Lewy-Body dementia, post-traumatic neurodegeneration, and chronic traumatic encephalopathy.

In another embodiment, the neurodegenerative disorder is not characterized by cognitive dysfunction. Non-limiting examples of neurodegenerative disorders not characterized by cognitive dysfunction include Parkinson's disease, amyotrophic lateral sclerosis (ALS), or multiple sclerosis (MS).

Also provided herein is a method for treating or preventing a condition or disorder characterized by cognitive dysfunction in a subject in need thereof comprising administering to the subject a therapeutically effective amount of a compound of the invention. Non-limiting examples of conditions or disorders characterized by cognitive dysfunction are schizophrenia, cognitive impairment associated with ischemic events, depression, and posttraumatic stress disorder (PTSD).

In another aspect, provided herein is a method for treating bipolar disorder in a subject in need thereof comprising administering a therapeutically effective amount of a compound of the invention.

In an aspect, provided herein is a method for treating or preventing Alzheimer's Disease in a subject in need thereof comprising administering to the subject a therapeutically effective amount of a compound of the invention.

In another aspect, provided herein is a method for treating Alzheimer's Disease in a subject in need thereof comprising administering to the subject a therapeutically effective amount of a compound of the invention.

In yet another aspect, provided herein is a method for enhancing cognition in a subject with Alzheimer's Disease comprising administering to the subject a compound of the invention.

In another aspect, provided herein is a method for enhancing memory in a subject with Alzheimer's Disease comprising administering to the subject a compound of the invention.

In an aspect, provided herein is a method for treating a neurodegenerative disorder in a subject in need thereof comprising administering to the subject a therapeutically effective amount of a compound of Formula I, or a pharmaceutically acceptable salt thereof.

Also provided herein is a method for treating or preventing a condition or disorder characterized by cognitive dysfunction in a subject in need thereof comprising administering to the subject a therapeutically effective amount of a compound of Formula I, or a pharmaceutically acceptable salt thereof.

In another aspect, provided herein is a method for treating bipolar disorder in a subject in need thereof comprising administering to the subject a therapeutically effective amount of a compound of Formula I, or a pharmaceutically acceptable salt thereof.

In an aspect, provided herein is a method for treating or preventing Alzheimer's Disease in a subject in need thereof comprising administering to the subject a therapeutically effective amount of a compound of Formula I, or a pharmaceutically acceptable salt thereof.

In another aspect, provided herein is a method for treating Alzheimer's Disease in a subject in need thereof comprising administering to the subject a therapeutically effective amount of a compound of Formula I, or a pharmaceutically acceptable salt thereof.

In yet another aspect, provided herein is a method for enhancing cognition in a subject with Alzheimer's Disease comprising administering to the subject a compound of Formula I, or a pharmaceutically acceptable salt thereof.

In another aspect, provided herein is a method for enhancing memory in a subject with Alzheimer's Disease comprising administering to the subject a compound of Formula I, or a pharmaceutically acceptable salt thereof.

In an aspect, provided herein is a method for treating a neurodegenerative disorder in a subject in need thereof comprising administering to the subject a therapeutically effective amount of a compound of Formula IA, or a pharmaceutically acceptable salt thereof.

Also provided herein is a method for treating or preventing a condition or disorder characterized by cognitive dysfunction in a subject in need thereof comprising administering to the subject a therapeutically effective amount of a compound of Formula IA, or a pharmaceutically acceptable salt thereof.

In another aspect, provided herein is a method for treating bipolar disorder in a subject in need thereof comprising administering to the subject a therapeutically effective amount of a compound of Formula IA, or a pharmaceutically acceptable salt thereof.

In an aspect, provided herein is a method for treating or preventing Alzheimer's Disease in a subject in need thereof comprising administering to the subject a therapeutically effective amount of a compound of Formula IA, or a pharmaceutically acceptable salt thereof.

In another aspect, provided herein is a method for treating Alzheimer's Disease in a subject in need thereof comprising administering to the subject a therapeutically effective amount of a compound of Formula IA, or a pharmaceutically acceptable salt thereof. In yet another aspect, provided herein is a method for enhancing cognition in a subject with Alzheimer's Disease comprising administering to the subject a compound of Formula IA, or a pharmaceutically acceptable salt thereof.

In another aspect, provided herein is a method for enhancing memory in a subject with Alzheimer's Disease comprising administering to the subject a compound of Formula IA, or a pharmaceutically acceptable salt thereof.

In an aspect, provided herein is a method for treating a neurodegenerative disorder in a subject in need thereof comprising administering to the subject a therapeutically effective amount of a compound of Formula II, or a pharmaceutically acceptable salt thereof.

Also provided herein is a method for treating or preventing a condition or disorder characterized by cognitive dysfunction in a subject in need thereof comprising administering to the subject a therapeutically effective amount of a compound of Formula II, or a pharmaceutically acceptable salt thereof.

In another aspect, provided herein is a method for treating bipolar disorder in a subject in need thereof comprising administering to the subject a therapeutically effective amount of a compound of Formula II, or a pharmaceutically acceptable salt thereof.

In an aspect, provided herein is a method for treating or preventing Alzheimer's Disease in a subject in a subject in need thereof comprising administering to the subject a therapeutically effective amount of a compound of Formula II, or a pharmaceutically acceptable salt thereof.

In another aspect, provided herein is a method for treating Alzheimer's Disease in a subject in need thereof comprising administering to the subject a therapeutically effective amount of a compound of Formula II, or a pharmaceutically acceptable salt thereof.

In yet another aspect, provided herein is a method for enhancing cognition in a subject with Alzheimer's Disease comprising administering to the subject a compound of Formula II, or a pharmaceutically acceptable salt thereof.

In another aspect, provided herein is a method for enhancing memory in a subject with Alzheimer's Disease comprising administering to the subject a compound of Formula II, or a pharmaceutically acceptable salt thereof.

In an aspect, provided herein is a method for treating a neurodegenerative disorder in a subject in need thereof comprising administering to the subject a therapeutically effective amount of Compound 1, or a pharmaceutically acceptable salt thereof.

Also provided herein is a method for treating or preventing a condition or disorder characterized by cognitive dysfunction in a subject in need thereof comprising administering to the subject a therapeutically effective amount of Compound 1, or a pharmaceutically acceptable salt thereof.

In another aspect, provided herein is a method for treating bipolar disorder in a subject in need thereof comprising administering to the subject a therapeutically effective amount of Compound 1, or a pharmaceutically acceptable salt thereof.

In an aspect, provided herein is a method for treating or preventing Alzheimer's Disease in a subject in a subject in need thereof comprising administering to the subject a therapeutically effective amount of Compound 1, or a pharmaceutically acceptable salt thereof.

In another aspect, provided herein is a method for treating Alzheimer's Disease in a subject in need thereof comprising administering to the subject a therapeutically effective amount of Compound 1, or a pharmaceutically acceptable salt thereof.

In yet another aspect, provided herein is a method for enhancing cognition in a subject with Alzheimer's Disease comprising administering to the subject Compound 1, or a pharmaceutically acceptable salt thereof.

In another aspect, provided herein is a method for enhancing memory in a subject with Alzheimer's Disease comprising administering to the subject Compound 1, or a pharmaceutically acceptable salt thereof.

In an aspect, provided herein is a method for treating a neurodegenerative disorder in a subject in need thereof comprising administering to the subject a therapeutically effective amount of Compound 2, or a pharmaceutically acceptable salt thereof.

Also provided herein is a method for treating or preventing a condition or disorder characterized by cognitive dysfunction in a subject in need thereof comprising administering to the subject a therapeutically effective amount of Compound 2, or a pharmaceutically acceptable salt thereof.

In another aspect, provided herein is a method for treating bipolar disorder in a subject in need thereof comprising administering to the subject a therapeutically effective amount of Compound 2, or a pharmaceutically acceptable salt thereof.

In an aspect, provided herein is a method for treating or preventing Alzheimer's Disease in a subject in a subject in need thereof comprising administering to the subject a therapeutically effective amount of Compound 2, or a pharmaceutically acceptable salt thereof.

In another aspect, provided herein is a method for treating Alzheimer's Disease in a subject in need thereof comprising administering to the subject a therapeutically effective amount of a compound of Compound 2, or a pharmaceutically acceptable salt thereof.

In yet another aspect, provided herein is a method for enhancing cognition in a subject with Alzheimer's Disease comprising administering to the subject Compound 2, or a pharmaceutically acceptable salt thereof.

In another aspect, provided herein is a method for enhancing memory in a subject with Alzheimer's Disease comprising administering to the subject Compound 2, or a pharmaceutically acceptable salt thereof.

In an aspect, provided herein is a method for treating a neurodegenerative disorder in a subject in need thereof comprising administering to the subject a therapeutically effective amount of Compound 9, or a pharmaceutically acceptable salt thereof.

Also provided herein is a method for treating or preventing a condition or disorder characterized by cognitive dysfunction in a subject in need thereof comprising administering to the subject a therapeutically effective amount of Compound 9, or a pharmaceutically acceptable salt thereof.

In another aspect, provided herein is a method for treating bipolar disorder in a subject in need thereof comprising administering to the subject a therapeutically effective amount of Compound 9, or a pharmaceutically acceptable salt thereof.

In an aspect, provided herein is a method for treating or preventing Alzheimer's Disease in a subject in a subject in need thereof comprising administering to the subject a therapeutically effective amount of Compound 9, or a pharmaceutically acceptable salt thereof.

In another aspect, provided herein is a method for treating Alzheimer's Disease in a subject in need thereof comprising administering to the subject a therapeutically effective amount of Compound 9, or a pharmaceutically acceptable salt thereof.

In yet another aspect, provided herein is a method for enhancing cognition in a subject with Alzheimer's Disease comprising administering to the subject Compound 9, or a pharmaceutically acceptable salt thereof.

In another aspect, provided herein is a method for enhancing memory in a subject with Alzheimer's Disease comprising administering to the subject Compound 9, or a pharmaceutically acceptable salt thereof.

Provided herein is a method for treating or preventing Alzheimer's disease in a subject comprising administering to the subject a therapeutically effective amount of a histone deacetylase 1/2 (HDAC1/2) selective inhibitor.

Provided herein is a method for enhancing cognition in a subject with Alzheimer's Disease comprising administering to the subject a histone deacetylase 1/2 (HDAC1/2) selective inhibitor.

Provided herein is a method for enhancing memory in a subject with Alzheimer's Disease comprising administering to the subject a histone deacetylase 1/2 (HDAC1/2) selective inhibitor.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In certain embodiments, the invention provides a method for treating of any of the disorders described herein, wherein the subject is a human.

The following examples further illustrate aspects of the present invention. However, they are in no way a limitation of the teachings or disclosure of the present invention as set forth herein.

### EXAMPLES

### Example 1: Synthesis of Compounds 1 and 2

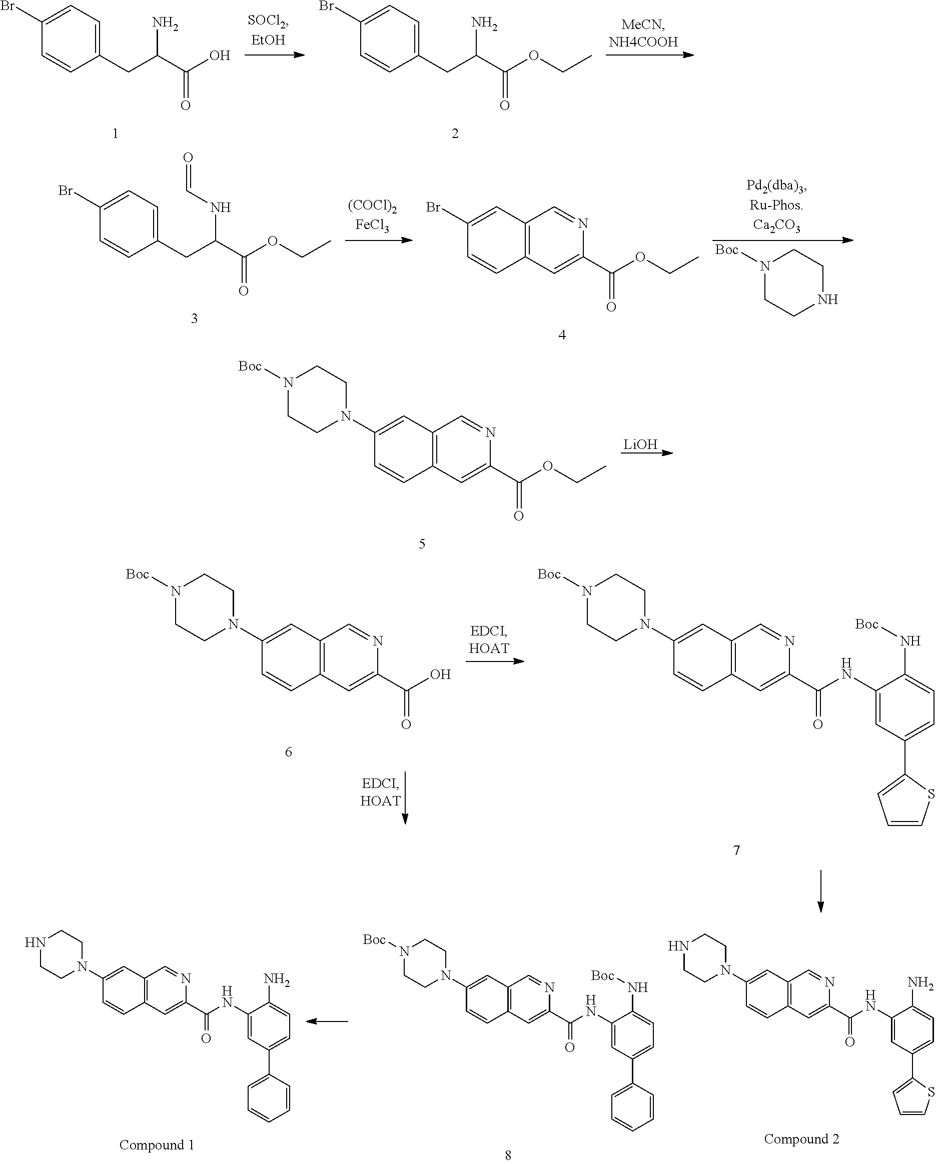

Step 1:

To 2-amino-3-(4-bromophenyl)propanoic acid (50 g, 0.2 mol) in ethanol (1 L) was added dropwise SOCl₂ (30 ml) at 0° C., after addition was completed, the mixture was refluxed for overnight. Stop the reaction, the mixture was evaporated to be dry, to the residue was added EA (500 ml) and saturated aqueous of NaHCO₃ (500 ml), the organic layer was separated out, the water was extracted with EA (200 ml), the combined organic layer was washed by brine and dried with Na₂SO₄, evaporated to be dry, the residue was used at next step without further purification. 45 g yellow solid was obtained. Yield: 81%. LCMS: 99% UV-214, [M+H]: 272.

Step 2:

To a flask containing ethyl 2-amino-3-(4-bromophenyl)propanoate (45 g, 166 mmol) in CH₃CN (500 ml) was added HCOONH₄ (95 g, 1.5 mol) at r.t., followed by stirring at 90° C. overnight. After the solvent was evaporated, to the residue was added water (1 L) and then extracted by EA (300 ml*3). The combined organic layer was washed with brine, dried by anhydrous Na₂SO₄, and concentrated in vacuo. The resulting residue was purified by silica gel chromatography (Elution: From PE/EA=4:1 to 3:1) to afford ethyl 3-(4-bromophenyl)-2-formamidopropanoate as a red solid (39 g, yield: 79.6%). LCMS: 92% UV-214, [M+H]: 300.

Step 3:

To a mixture of ethyl-3-(4-bromophenyl)-2-formamidopropanoate (39 g, 130 mmol) in DCM (500 mL) was added (CO)₂Cl₂ (18 g, 143 mmol). The reaction mixture was stirred at r.t. for 30 min. Then the reaction solution was cooled to 0° C. and FeCl₃ (26 g, 163 mmol) was added into the solution, followed by stirring at r.t overnight. After the solution was extracted by CH₂Cl₂, the combined organic layers were concentrated in vacuo to afford a black oil. The oil was dissolved in the EtOH (300 ml), and concentrated H₂SO₄ was added into the solution and refluxed at 80° C. overnight. The solution was poured into saturated NaHCO₃ and extracted by EA. After drying with anhydrous Na₂SO₄, the solution was concentrated in vacuo, and the residue was purified by silica gel chromatography (PE/EA=8:1 to 5:1) to afford ethyl 7-bromoisoquinoline-3-carboxylate as a yellow solid (5.2 g, yield: 15%). LCMS: 95% UV-214, [M+H]: 280.

Step 4:

A mixture of intermediate 4 (5.2 g, 18.6 mmol), tert-butyl piperazine-1-carboxylate (4.2 g, 22.4 mmol), Pd₂(dba)₃ (920 mg, 1 mmol), RuPhos(950 mg, 2 mmol) and Cs₂CO₃(12 g, 37 mmol) in dioxane (150 mL) was stirred at 100° C. under N₂ atmosphere for 4 hr. The mixture was cooled, filtered, and concentrated to obtain a residue, which was purified by combiflash (Elution, PE: EA=40%) to afford intermediate 5 (4.5 g, 63%) as yellow solid. LCMS: 98% UV-214, [M+H]: 386.

Step 5:

A solution of intermediate 5 (4.5 g, 11.7 mmol) in MeOH (50 mL) and THF (50 ml) was added the aqueous of NaOH (2.4 g, 58.5 mmol in water 30 ml and stirred at 60° C. for 3 h. The mixture was concentrated to get a residue, to the residue was added water (100 ml), and then adjusted pH to about 6 using HCl (0.5N) carefully, yellow solid was separated out, the mixture was filtered, the solid was washed by water (50 ml) and dried to give intermediate 6 as yellow solid (3.5 g, yield: 83%). LCMS: 98.9% UV-214, [M+H]: 35.

Step 6:

A mixture of intermediate 6 (1.5 g, 4.2 mmol), tert-butyl 3-aminobiphenyl-4-ylcarbamate (1.43 g, 5 mmol), HATU (2.4 g, 6.3 mmol), DIPEA (1.1 g, 8.4 mmol) in DCM (100 ml) was stirred at room temperature for 1 hr. the mixture was evaporated to be dry, the residue was purified by silica gel column chromatography (DCM:MeOH=20:1) to give intermediate 8 as yellow solid (1.6 g, Yield: 62%). LCMS: 97% UV-214, [M+H]: 624.

Step 7:

A mixture of intermediate 6 (2 g, 5.6 mmol), tert-butyl(2-amino-4-(thiophen-2-yl)phenyl)carbamate (1.95 g, 6.7 mmol), HATU (3.2 g, 8.4 mmol), DIPEA (1.5 g, 11.2 mmol) in DCM (100 ml) was stirred at room temperature for 1 hr. the mixture was evaporated to be dry, the residue was purified by silica gel column chromatography (DCM:MeOH=20:1) to give intermediate 7 as pale-white solid (1.8 g, Yield: 50%). LCMS: 100% UV-214, [M+H]: 630.

Step 8 (Synthesis of Compound 2):

To a solution of intermediate 7 (1.8 g, 2.86 mmol) in DCM (100 ml) was added 4N HCl in dioxane (30 ml), the mixture was stirred at room temperature for 2 hr and stopped. The mixture was filtered, the solid was washed by ether (20 ml) and dried to give Compound 2 (1.1 g, HCl salt, yield: 62%) as yellow solid. LCMS: 100% UV-214, [M+H]: 430. ¹H NMR (500 MHz, MeOD): 3.506 (t, 4H), 3.871 (t, 4H), 7.182 (dd, 1H), 7.536 (dd, 1H), 7.576 (dd, 1H), 7.603 (d, 1H), 7.818 (dd, 1H), 7.894 (d, 1H), 7.939 (d, 1H), 8.133 (dd, 1H), 8.338 (d, 1H), 9.110 (s, 1H), 9.551 (s, 1H).

Step 9 (Synthesis of Compound 1):

To a solution of intermediate 8 (1.6 g, 2.57 mmol) in DCM (100 ml) was added 4N HCl in dioxane (30 ml), the mixture was stirred at room temperature for 2 hr and stopped. The mixture was filtered, the solid was washed by ether (20 ml) and dried to give Compound 1 (1.29 g, HCl salt, yield: 83%) as yellow solid. LCMS: 100% UV-214, [M+H]: 424. ¹H NMR (500 MHz, MeOD): 3.506 (t, 4H), 3.881 (t, 4H), 7.453 (t, 1H), 7.527 (t, 2H), 7.671 (d, 1H), 7.737 (d, 2H), 7.811 (dd, 1H), 7.924 (m, 2H), 8.146 (dd, 1H), 8.343 (d, 1H), 9.171 (s, 1H), 9.574 (s, 1H).

### Example 2: HDAC Enzyme Assays

Compounds for testing were diluted in DMSO to 50 fold the final concentration and a ten point three fold dilution series was made. The compounds were diluted in assay buffer (50 mM HEPES, pH 7.4, 100 mM KCl, 0.001% Tween-20, 0.05% BSA, 20 μM TCEP) to 6 fold their final concentration. The HDAC enzymes (purchased from BPS Biosciences) were diluted to 1.5 fold their final concentration in assay buffer. The tripeptide substrate and trypsin at 0.05 μM final concentration were diluted in assay buffer at 6 fold their final concentration. The final enzyme concentrations used in these assays were 3.3 ng/ml (HDAC1), 0.2 ng/ml (HDAC2) and 0.08 ng/ml (HDAC3). The final substrate concentrations used were 16 μM (HDAC1), 10 μM (HDAC2) and 17 μM (HDAC3). Five μl of compound and 20 μl of enzyme were added to wells of a black, opaque 384 well plate in duplicate. Enzyme and compound were incubated together at room temperature for 24 hours. Five μl of substrate was added to each well, the plate was shaken for 60 seconds and placed into a Victor 2 microtiter plate reader. The development of fluorescence was monitored for 60 min and the linear rate of the reaction was calculated. The IC₅₀ was determined using Graph Pad Prism by a four parameter curve fit. The IC₅₀ values for Compounds 1-7 are shown below in Table 2.

TABLE 2

| | IC₅₀ (nM) | | |
|---|---|---|---|
| Compound | HDAC1 | HDAC2 | HDAC3 |
| Compound 1 | 5 | 13 | 84 |
| Compound 2 | 3 | 10 | 43 |
| Compound 3 | 7 | 8 | 74 |
| Compound 4 | 6 | 8 | 182 |
| Compound 5 | 2 | 6 | 19 |
| Compound 6 | 5 | 19 | 121 |
| Compound 7 | 3 | 10 | 66 |

### Example 3. Histone Acetylase 1/2 (HDAC1/2) Inhibitor Compound 1 Improves Cognition in APPSwDI/NOS2−/− Model of Alzheimer's Disease

The ability of HDAC1/2 inhibitor Compound 1 to improve cognitive deficits of APPSwDI/NOS2−/− transgenic mice was tested in the radial arm water maze (RAWM). APPSwDI/NOS2−/− mice received Compound 1 daily for 4 weeks and the drug was found to be very well tolerated in all animals.

During MWM training period transgenic mice that received the drug showed significantly better learning behavior and were able to remember the target position a lot better compared to the transgenic animals that received the vehicle. The learning behavior of Compound 1-treated transgenic mice was on par with that of the non-transgenic controls. Transgenic animals that received Compound 1 showed an improved escape latency time as well as a decrease in the number of errors when searching for the escape platform. These results suggest that HDAC1/2 inhibitor Compound 1 has positive effect on learning and potentially early consolidation processes in an Alzheimer's disease model and support further development of HDAC1/2 inhibitors as cognitive enhancers in AD.

I. Study Design and Method

The Study was performed on APPSwDI/NOS2−/− transgenic (mouse model of Alzheimer's disease) as well as non-transgenic mice, bred at Charles River (Finland). Animals for the main study are 10.5 months old at start. A total number of 45 transgenic (divided into 22 untreated and 23 treated) and 25 non-transgenic controls are used for the study

Transgenic mice are allocated to two different treatment groups, either treated with vehicle (20% HP-β-CD in water) or Compound 1 (dosage 10 mg/kg) administered orally via gavage daily for 4 weeks total. The application volume is 10 ml/kg/dose whereby each animal is weighed once a week and receives a volume according to its individual actual weight.

Spatial learning capacities of all animals were tested in the Radial Arm Water Maze (RAWM). Briefly a six arm maze was submerged in a pool of water, and a platform was placed at the end of one arm. The mouse received 15 trials per day for 2 days and on each trial was started in a different arm while the arm containing the platform remained the same for each mouse. The first 10 trials were considered training and alternated between a visible and a hidden platform. The final trials for day 1 and all trials on day 2 were tested with a hidden platform. The number of errors (incorrect arm entries) were counted over a 1 min period. The errors were averaged over six trials resulting in 5 blocks for the 2 day period. Latency to find the platform was also recorded, and averaged similarly as the errors.

II. Results

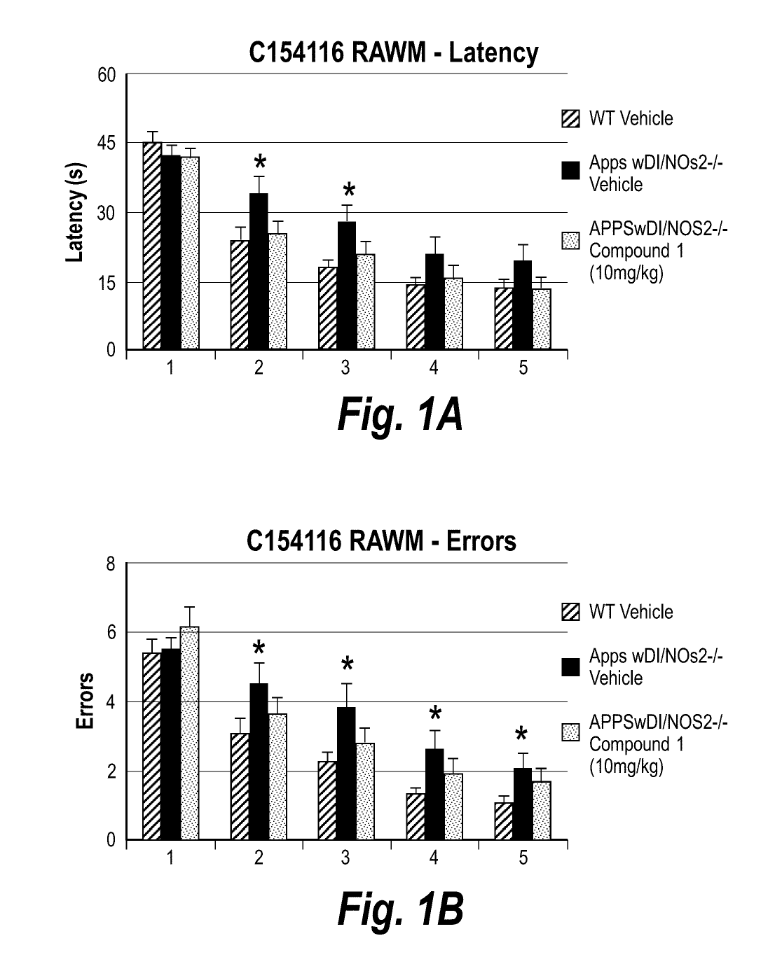

There were no differences in body weight between the treatment groups. Radial arm maze latency and number of errors were significantly increased in APPSwDI/NOS2−/− vehicle treated mice compared to WT vehicle mice (p<0.05) (FIG. 1 A-B). Treatment with Compound 1 10 mg/kg reversed the increased latency and number of errors in APPSwDI/NOS2−/− mice compared to vehicle treated APPSwDI/NOS2−/− (FIG. 1 A-B).

### Example 4. Histone Acetylase 1/2 (HDAC1/2) Inhibitor Compound 1 Improves Cognition in Aged Mice

The ability of HDAC1/2 inhibitor Compound 1 to improve cognitive deficits in aged mice was tested by InterVivo Solutions (Toronto, Canada). Young (3 months old) and aged (18 months old) mice were compared in a Morris Water Maze to measure differences in learning and memory.

I. Study Design and Method

Mice were first placed in a pool of water and trained to swim to a visible flagged platform (the cued task). The mice received 3 trails per day for 5 days and the time it took for the mice to find the platform was timed. Young mice and aged mice performed equally well at this task, indicating there was no difference in swimming speed between the two cohorts of mice.

Next, mice were trained in a place task, in which the platform is submerged and hidden with flags to indicate the fixed location of the platform. The mice were trained for 4 days with 3 trials per day.

II. Results

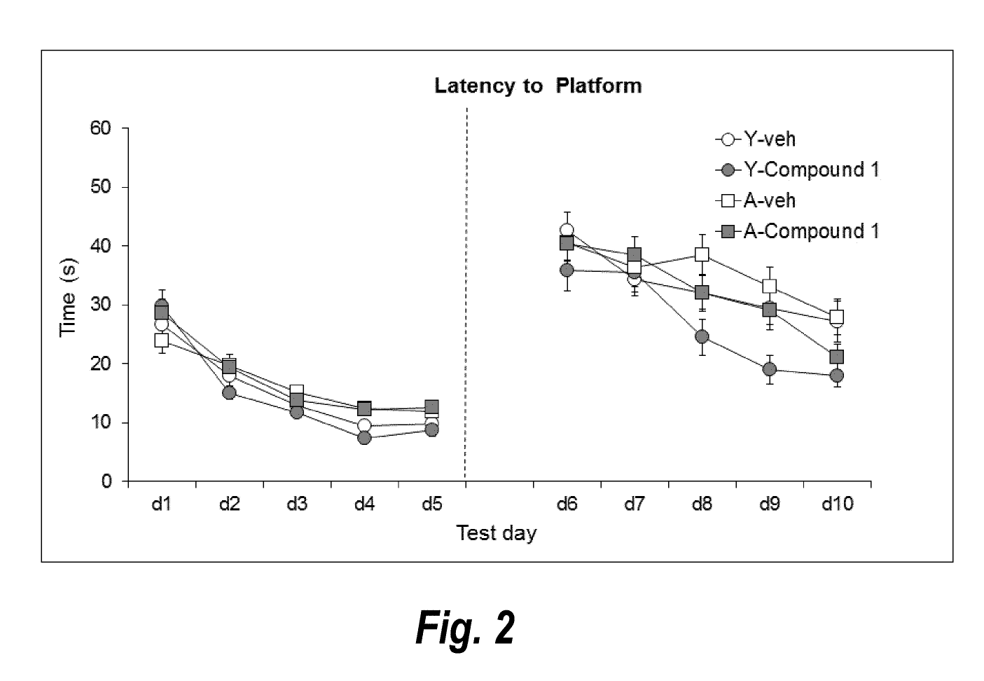

In the place task the young mice improved their escape latency on days 3 and 4 as compared to days 1 and 2, while aged mice improved on days 4 and 5. Treatment of the aged mice with compound 1 (10 mg/kg/day, 4 weeks) caused the aged cohort to learn at a rate greater than the young untreated cohort. Treatment of the young mice with compound 1 caused a dramatic improvement in learning rate, resulting in a 25% reduction in escape latency as compared to untreated mice (FIG. 2).

### Example 5. Pharmacokinetic Parameters of Compounds 1, 2, 3, and 4

Male C57Bl/6 mice were fasted overnight. Compounds of the invention were dissolved in dimethyl acetamide at 10 times the final concentration, then Solutol HS 15 (BASF) was added to a final concentration of 10%. Finally 80% saline was added and vortexed to achieve a clear solution. Fifteen animals were injected via the tail vein with 1 mg/kg compound. Blood was collected via retro orbital puncture into K2EDTA tubes at 5 minutes, 15 minutes, 30 minutes, 1 hour, 2 hours, 4 hours, 8 hours and 24 hours after dosing. The blood was centrifuged at 2000 g for 5 minutes at 4° C. to obtain plasma. The plasma was extracted with acetonitrile and the level of compound was analyzed by LC/MS/MS. The level of compound in plasma was calculated from a standard curve in mouse plasma. For brain levels, animals were sacrificed at 5 minutes, 15 minutes, 1 hour, 4 hours and 24 hours. Brains were removed and homogenized in acetonitrile and extracted. The level of compound in brain was calculated from a standard curve in mouse brain tissue. The IV clearance and area under the curve were calculated using WinNonLin software. The ratio of area under the curve for plasma and brain samples was calculated to produce the brain/plasma ratio.

TABLE 3
Pharmacokinetic parameters of Compounds 1, 2, 3, and 4 in plasma after
IV administration at 1 mg/kg in male C57BL/6 mice (in PK cassette)

| PK parameters | Compound | Compound | Compound | Compound |
|---|---|---|---|---|
| (unit) | 1 | 2 | 3 | 4 |
| CL (L/hr/kg) | 0.333 | 0.619 | 1.25 | 0.262 |
| Vₛₛ (L/kg) | 3.47 | 4.31 | 6.82 | 2.28 |
| T_{1/2} (hr) | 7.80 | 5.29 | 5.68 | 7.71 |
| AUCₗₐₛₜ | 2683 | 1556 | 770 | 3472 |
| (hr * ng/mL) | | | | |
| AUC_{INF} | 3006 | 1616 | 799 | 3819 |
| (hr * ng/mL) | | | | |
| MRT_{INF} (hr) | 10.4 | 6.97 | 5.45 | 8.72 |

TABLE 4
Pharmacokinetic parameters of Compounds 1, 2, 3, 4 and 9 in brain after
IV administration at 1 mg/kg in male C57BL/6 mice

| PK | | | | | |
|---|---|---|---|---|---|
| parameters | | | | | |
| (unit) | Comp. 1 | Comp. 2 | Comp. 3 | Comp. 4 | Comp. 9 |
| Tₘₐₓ (hr) | 1.00 | 1.00 | 0.250 | 0.250 | 1.00 |
| Cₘₐₓ (ng/g) | 675 | 783 | 126 | 145 | 1002 |
| T_{1/2} (hr) | 9.12 | 9.30 | 8.63 | 1.66 | 4.4 |
| AUCₗₐₛₜ | 10505 | 11474 | 1237 | 319 | 9444 |
| (hr * ng/g) | | | | | |
| AUC_{INF} | 12187 | 13461 | 1439 | 390 | 9607 |
| (hr * ng/g) | | | | | |
| AUC_{brain}/ | 405 | 833 | 180 | 10 | 1026 |
| AUCₚₗₐₛₘₐ | | | | | |
| (%) | | | | | |

The very high brain/plasma AUC ratios found for Compounds 1, 2 and 9 suggest these compounds are highly brain penetrant compounds.

### Example 6. Pharmacokinetic Parameters of Compound 1 after IP and PO Administration at 3 and 10 mg/kg in Male C57BL/6 Mice (Discrete PK)

Male C57Bl/6 mice were fasted overnight. Compound 1 was dissolved in 20% HydroxyPropyl-β-Cyclodextran in water. Fifteen animals per experimental arm were dosed by intraperitoneal injection or by oral gavage. Blood was collected via retro orbital puncture into K2EDTA tubes at 5 minutes, 15 minutes, 30 minutes, 1 hour, 2 hours, 4 hours, 8 hours and 24 hours after dosing. The blood was centrifuged at 2000 g for 5 minutes at 4° C. to obtain plasma. The plasma was extracted with acetonitrile and the level of compound was analyzed by LC/MS/MS. The level of compound in plasma was calculated from a standard curve in mouse plasma. For brain levels, animals were sacrificed at 15 minutes, 1 hour, 4 hours, 8 hours and 24 hours. Brains were removed and homogenized in acetonitrile and extracted. The level of compound in brain was calculated from a standard curve in mouse brain tissue. The maximum concentration, half-life and area under the curve were calculated using WinNonLin software. The ratio of area under the curve for plasma and brain samples was calculated to produce the brain/plasma ratio.

TABLE 5
Summary of plasma PK parameters of Compound 1 after IP or PO
administration in male C57BL/6 mice

| PK | | IP | IP | PO | PO |
|---|---|---|---|---|---|
| parameters | Unit | 3 mg/kg | 10 mg/kg | 3 mg/kg | 10 mg/kg |
| Tₘₐₓ | hr | 0.500 | 0.250 | 2.00 | 1.00 |
| Cₘₐₓ | ng/mL | 318 | 1015 | 135 | 561 |
| Terminal t_{1/2} | hr | 3.88 | 4.20 | 3.44 | 4.08 |
| AUCₗₐₛₜ | hr * ng/mL | 1306 | 4960 | 1472 | 4987 |
| AUC_{INF} | hr * ng/mL | 1320 | 5043 | 1482 | 5060 |

TABLE 6
Summary of brain PK parameters of Compound 1 after IP or PO
administration in male C57BL/6 mice

| PK | | IP | IP | PO | PO |
|---|---|---|---|---|---|
| parameters | Unit | 3 mg/kg | 10 mg/kg | 3 mg/kg | 10 mg/kg |
| Tₘₐₓ | hr | 4.00 | 4.00 | 4.00 | 8.00 |
| Cₘₐₓ | ng/g | 1487 | 5318 | 743 | 3653 |
| Terminal t_{1/2} | hr | 5.87 | 7.13 | 5.80 | 6.76 |
| AUC_{last-brain} | hr * ng/g | 19737 | 78978 | 10019 | 53716 |
| AUC_{INF-brain} | hr * ng/g | 21015 | 87609 | 10651 | 58481 |
| AUC_{brain}/ | % | 1512 | 1592 | 681 | 1077 |
| AUCₚₗₐₛₘₐ | | | | | |

The high brain/plasma AUC ratios indicate that Compound 1 is a highly brain penetrant compound.

### Example 7. Comparison of Brain Penetrance Between Quinolines and Isoquinolines

Comparator B (see Table 7) and Compound 2 were dosed in C57Bl/6 mice at 5 mg/kg in 10% DMAC/10% Solutol HS15/80% saline. Comparator A (see Table 7) was dosed at 5 mg/kg in 5% dextrose/water. All compounds were dosed IP. Mice were sacrificed at 5, 15, 30 minutes and 1, 2, 4, 8 and 24 hours after dosing. Plasma concentration of compound was determined at all time points by LC/MS/MS. Brain concentration was determined at 1, 4, 8 and 24 hours after dosing. The AUC was calculated using WinNonLin software. The results are shown in Table 7, where plasma AUC is expressed as hr*ng/mL and brain AUC is expressed as hr*ng/g.

TABLE 7

| Compound | Structure | AUC plasma | AUC brain | ratio |
|---|---|---|---|---|
| Comparator A (Quinoline) | 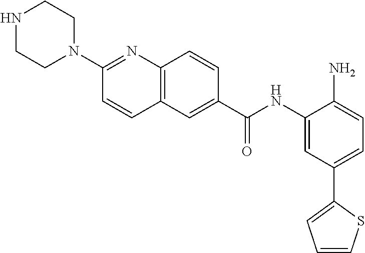 [SMILES: Nc1ccc(-c2cccs2)cc1NC(=O)c1ccc2nc(N3CCNCC3)ccc2c1] | 12899 | 2092 | 0.16 |
| Comparator B (Quinoline) | 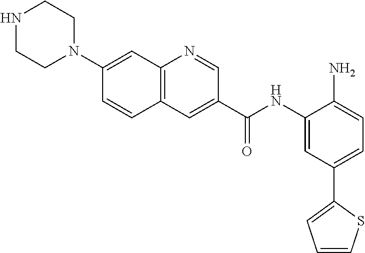 [SMILES: Nc1ccc(-c2cccs2)cc1NC(=O)c1cnc2cc(N3CCNCC3)ccc2c1] | 10777 | 655 | 0.06 |
| Compound 2 | See Table 1 | 3226 | 23283 | 7.2 |
| (Isoquinoline) | | | | |

1. A compound of Formula I 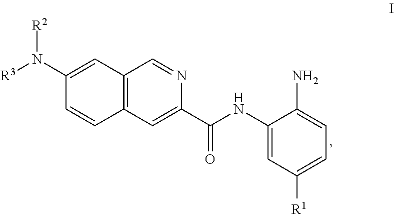
or a pharmaceutically acceptable salt thereof,
wherein
R¹ is aryl or heteroaryl;
R² is H, C₁-C₆-alkyl, or C₁-C₆-alkyl-N(R^{a})₂;
R³ is H, C₁-C₆-alkyl, C₁-C₆-alkyl-N(R^{a})₂, or C(O)R^{b};
or R² and R³, together with the N atom to which they are attached, optionally form a 5 or 6 membered heterocycloalkyl ring, wherein the heterocycloalkyl ring optionally contains a —N(R^{c})— moiety and wherein the heterocycloalkyl ring optionally contains a —C(O)— moiety;
each R^{a} is independently H or C₁-C₆-alkyl;
R^{b} is C₁-C₆-alkyl, C₁-C₆-alkyl-N(R^{d})₂, or a 5 or 6 membered heterocycloalkyl, wherein the heterocycloalkyl is optionally substituted by C₁-C₆-alkyl;
R^{c} is H or C₁-C₆-alkyl; and
each R^{d} is independently H or C₁-C₆-alkyl.

2. The compound of claim 1, wherein R¹ is phenyl, thiophenyl, or pyridinyl.

3. The compound of claim 1, wherein R² is H and R³ is C₁-C₆-alkyl-N(R^{a})₂.

4. The compound of claim 1, wherein
R² is H or C₁-C₆-alkyl;
R³ is C₁-C₆-alkyl-N(R^{a})₂ or C(O)R^{b};
or R² and R³, together with the N atom to which they are attached, optionally form a 5 or 6 membered heterocycloalkyl ring, wherein the heterocycloalkyl ring optionally contains a —C(O)— moiety and wherein the heterocycloalkyl ring optionally contains a —N(R^{c})— moiety.

5. The compound of claim 1, wherein R² and R³, together with the N atom to which they are attached, form a 5 or 6 membered heterocycloalkyl ring, wherein the heterocycloalkyl ring optionally contains a —N(R^{c})— moiety and wherein the heterocycloalkyl ring optionally contains a —C(O)— moiety.

6. The compound of claim 1, which is a compound of Formula II 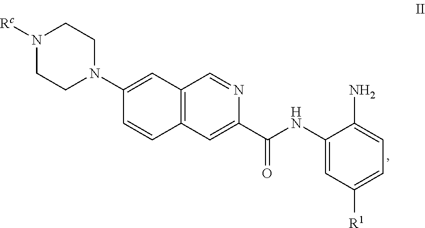
or a pharmaceutically acceptable salt thereof,
wherein
R¹ is aryl or heteroaryl; and
R^{c} is H or C₁-C₆-alkyl.

7. The compound of claim 6, wherein R¹ is phenyl, thiophenyl, or pyridinyl.

8. The compound of claim 6, wherein R¹ is phenyl or thiophenyl.

9. The compound of claim 6, wherein R^{c} is H.

10. A compound of Formula IA 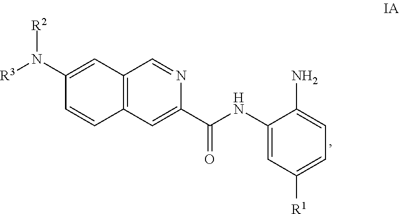
or a pharmaceutically acceptable salt thereof,
wherein
R¹ is aryl that is substituted one or more times with halo;
R² is H, C₁-C₆-alkyl, or C₁-C₆-alkyl-N(R^{a})₂;
R³ is H, C₁-C₆-alkyl, C₁-C₆-alkyl-N(R^{a})₂, or C(O)R^{b};
or R² and R³, together with the N atom to which they are attached, optionally form a 5 or 6 membered heterocycloalkyl ring, wherein the heterocycloalkyl ring optionally contains a —N(R^{c})— moiety and wherein the heterocycloalkyl ring optionally contains a —C(O)— moiety;
each R^{a} is independently H or C₁-C₆-alkyl;
R^{b} is C₁-C₆-alkyl, C₁-C₆-alkyl-N(R^{d})₂, or a 5 or 6 membered heterocycloalkyl, wherein the heterocycloalkyl is optionally substituted by C₁-C₆-alkyl;
R^{c} is H or C₁-C₆-alkyl; and
each R^{d} is independently H or C₁-C₆-alkyl.

11. A compound selected from 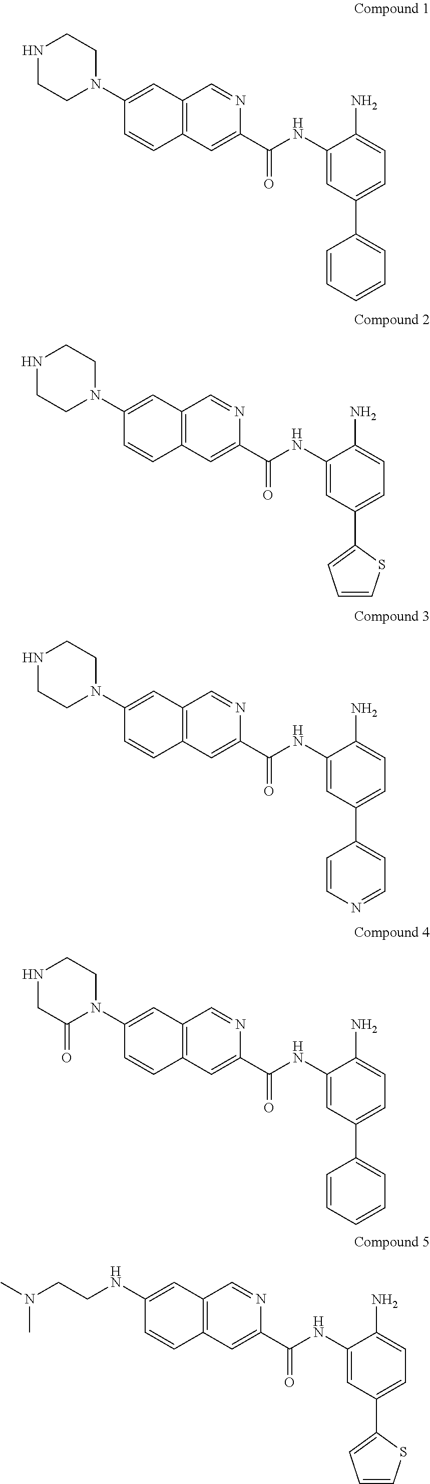 [SMILES: Nc1ccc(-c2ccccc2)cc1NC(=O)c1cc2ccc(N3CCNCC3)cc2cn1 ; Nc1ccc(-c2cccs2)cc1NC(=O)c1cc2ccc(N3CCNCC3)cc2cn1 ; Nc1ccc(-c2ccncc2)cc1NC(=O)c1cc2ccc(N3CCNCC3)cc2cn1 ; Nc1ccc(-c2ccccc2)cc1NC(=O)c1cc2ccc(N3CCNCC3=O)cc2cn1 ; CN(C)CCNc1ccc2cc(C(=O)Nc3cc(-c4cccs4)ccc3N)ncc2c1] 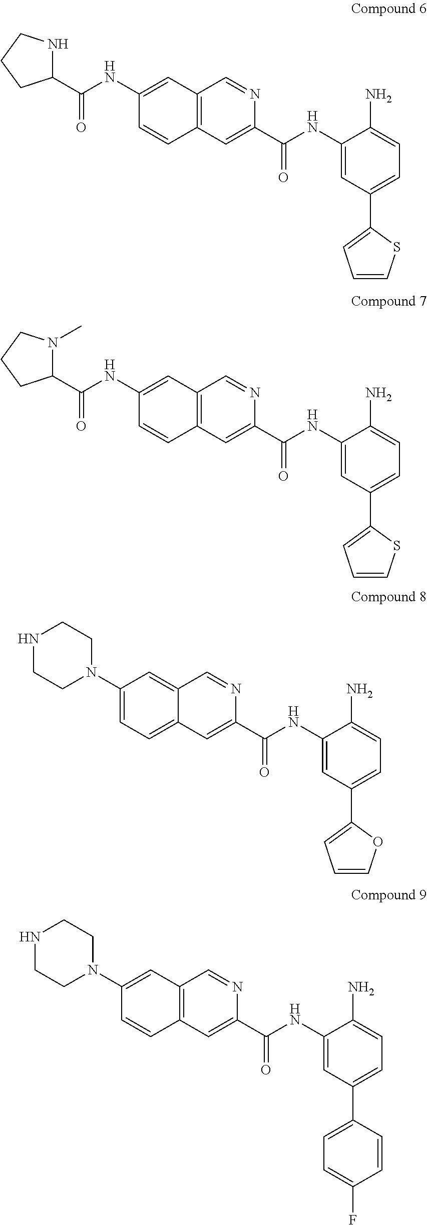 [SMILES: Nc1ccc(-c2cccs2)cc1NC(=O)c1cc2ccc(NC(=O)C3CCCN3)cc2cn1 ; CN1CCCC1C(=O)Nc1ccc2cc(C(=O)Nc3cc(-c4cccs4)ccc3N)ncc2c1 ; Nc1ccc(-c2ccco2)cc1NC(=O)c1cc2ccc(N3CCNCC3)cc2cn1 ; Nc1ccc(-c2ccc(F)cc2)cc1NC(=O)c1cc2ccc(N3CCNCC3)cc2cn1]
and pharmaceutically acceptable salts thereof.

12. The compound of claim 1, which is 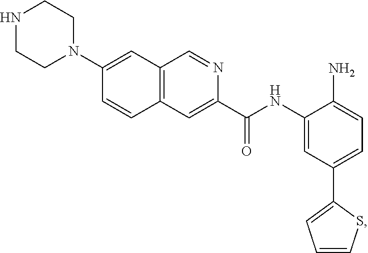 [SMILES: Nc1ccc(-c2cccs2)cc1NC(=O)c1cc2ccc(N3CCNCC3)cc2cn1]
or a pharmaceutically acceptable salt thereof.

13. The compound of claim 1, which is 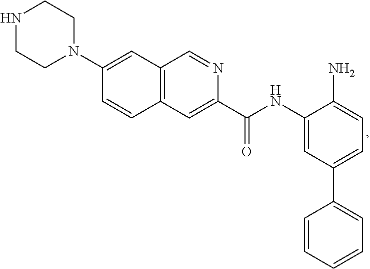 [SMILES: Nc1ccc(-c2ccccc2)cc1NC(=O)c1cc2ccc(N3CCNCC3)cc2cn1]
or a pharmaceutically acceptable salt thereof.

14. The compound of claim 10, which is 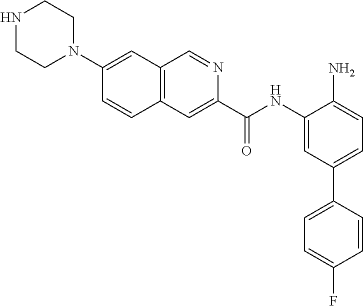 [SMILES: Nc1ccc(-c2ccc(F)cc2)cc1NC(=O)c1cc2ccc(N3CCNCC3)cc2cn1]
or a pharmaceutically acceptable salt thereof.

15. A pharmaceutical composition comprising a compound of claim 1 and at least one pharmaceutically acceptable carrier.

16. A method for selectively inhibiting histone deacetylase 1/2 (HDAC1/2) in a cell comprising contacting said cell with a compound of claim 1.

17. A method for treating a disease associated with HDAC1/2 activity in a subject in need thereof comprising administering to the subject a therapeutically effective amount of a compound of claim 1.

18. A method for treating a neurodegenerative disorder in a subject in need thereof comprising administering to the subject a therapeutically effective amount of a compound of claim 1.

19. (canceled)

20. (canceled)

21. (canceled)

22. (canceled)

23. (canceled)

24. (canceled)

25. (canceled)

26. A method for treating or preventing Alzheimer's Disease in a subject in need thereof comprising administering to the subject a therapeutically effective amount of a compound of claim 1.

27. (canceled)

28. (canceled)

29. (canceled)

30. A method for treating or preventing Alzheimer's disease in a subject comprising administering to the subject a therapeutically effective amount of a histone deacetylase 1/2 (HDAC1/2) selective inhibitor.

31. (canceled)

32. (canceled)

33. (canceled)

34. (canceled)

35. (canceled)
